(19) United States
(12) Patent Application Publication
(10) Pub. No.: US 2022/0370623 A1
(43) Pub. Date: Nov. 24, 2022

(54) Nanoparticles Comprising Prodrugs Stabilized by Albumin for Treatment of Cancer and Other Diseases

(71) Applicant: Luminus Biosciences, Inc., San Antonio, TX (US)

(72) Inventors: Ulagaraj Selvaraj, Carlsbad, CA (US); David Woody, New Braunfels, TX (US); John Henry Boatright, San Marcos, TX (US); Dong Wen, Cary, NC (US)

(21) Appl. No.: 17/774,749

(22) PCT Filed: Nov. 5, 2020
(86) PCT No.: PCT/US20/59182

§ 371 Date: May 5, 2022

Related U.S. Application Data
(60) Provisional application No. 62/931,048, filed on Nov. 5, 2019.

(51) Int. Cl. A61K 47/54; A61K 47/64; A61K 31/337; A61K 31/436

(52) U.S. Cl. CPC A61K 47/542; A61K 47/643; A61K 31/337; A61K 31/436; B82Y 5/00

(57) ABSTRACT

The present invention provides pharmaceutical compositions comprising solid nanoparticles, wherein the solid nanoparticles comprise i) an effective amount of a therapeutically active agent, wherein the therapeutically active agent is a substantially water insoluble prodrug; and ii) a biocompatible polymer.

### CROSS-REFERENCE TO RELATED APPLICATIONS

This application claims the benefit of U.S. Provisional Appl. No.: 62/931,048, filed Nov. 5, 2019, the entire contents of which are hereby incorporated by reference.

### FIELD OF THE INVENTION

The field of the invention relates to pharmaceutical formulations, in particular pharmaceutical nanoparticle compositions for the treatment of cancer and other diseases.

### SUMMARY OF THE INVENTION

In some aspects, the invention relates to drug formulations for the treatment of cancer, and other diseases comprising nanoparticles stabilized by human albumin. In some embodiments, the invention provides combination therapy options, comprising administration of therapeutic quantity of the prodrug nanoparticles stabilized by albumin.

The inventors discovered that drug molecules can be covalently conjugated with fatty acids to yield highly water insoluble prodrugs. The highly lipophilic prodrug can be combined with human albumin by a suitable process, resulting in a stable nanoparticle formulation.

A prodrug is defined as a derivative of an active drug, which is non-toxic and pharmacodynamically inert. However, following administration into the body, the prodrug can be transformed in vivo to a pharmacologically active drug. Examples of prodrug ester groups include docosahexaenoyl, eicosapentaenoyl, α-linolenoyl, oleyl, palmityl, stearyl, cholesteryl, cetostearyl, cetaryl, lauryl, decyl, undecyl, acetyl, propionyl, butyryl, pentanyl, hexanyl, heptanyl, octanyl, nonyl, decanyl, undecanyl, dodecanyl, and phthalyl. Other examples of suitable prodrug ester groups and external acids can be found in the book “Pro-drugs as Novel Delivery Systems,” by Higuchi and Stella, Vol. 14 of the American Chemical Society Symposium Series, American Chemical Society (1975).

In some instances, for example, when cabazitaxel, everolimus, docetaxel and similar drug molecules are combined with human albumin, nanoparticle formulations are formed. However, within a few hours these nanoparticle formulations undergo Ostwald ripening and result in micron size particles and are not suitable to develop as parenteral products. However, it has been discovered that when the lipophilic prodrugs of cabazitaxel, everolimus, docetaxel are combined with human albumin by a suitable process, stable prodrug nanoparticle stabilized by human albumin can be obtained. The Oswald ripening process is prevented in the nanoparticle prodrug stabilized by human albumin due to the highly lipophilic prodrug molecule.

The current invention involves improving many physicochemical, biopharmaceutical, and the clinical efficacy of various drugs using nanoparticle prodrugs. The applications of the prodrug are the same as the drug from which it is synthesized, however, it has enhanced therapeutic properties. The present invention is also directed to pharmaceutical compositions containing the same.

In some embodiments, the nanoparticle prodrug is designed to improve the safety and effectiveness of drug chemotherapy by delivering more therapeutic agent to tumor cells and less to healthy tissues where side effects often occur. In some embodiments, the prodrug is designed to maximize anticancer effects by targeting the tumor preferentially to normal tissue. For example, docosahexaenoic acid (DHA)—docetaxel or cabazitaxel or everolimus is a novel prodrug; DHA is a prevalent fatty acid, essential for normal human development and approved for exogenous administration by the European regulatory authorities and the World Health Organization. The nanoparticle prodrug dispersions prepared according to the present invention exhibit little or no particle growth mediated by Ostwald ripening.

In some embodiments, the formulation of prodrug is substantially free of toxic solvents such as ethanol and polyethylene glycol and surfactants such as cremophor EL and polysorbate 80; the standard vehicles used to formulate such highly lipophilic molecules. In some embodiments, the finished lyophilized product can be reconstituted in 0.9% saline to a maximum concentration of 5 mg/ml and administered intravenously over 30 minutes every week. Owing to the absence of surfactants, the use of steroid and antihistamine premedications, as well as non-PVC tubing and in-line filtration systems, are not required for drug administration.

In a further embodiment, the prodrug composition provided includes the drug and the fatty acid having a covalent bond to the drug wherein the drug is selected from the group consisting of: taxanes (paclitaxel, docetaxel, cabazitaxel, larotaxel, TPI-287, ortataxel, milataxel, BMS-184476, and others), camptothecins (topotecan, irinotecan, SN-38, S39625, and S38809), doxorubicin, eribulin, rapamycin, cytarabine, etoposide, podophyllotoxin, temozolomide, methotrexate, floxuridine, gemcitabine, mitomycin, riluzole, cladribine, melphalan, cidofovir, fulvestrant, melphalan, cannabinoids (cannabidiol, tetrahydrocannabinol, cannabinol, cannabigerol, tetrahydrocannabinolic acid, cannabidiolic acid, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran), aprepitant, morphine, hydrocodone, and others.

In one aspect, the invention provides a pharmaceutical composition comprising solid nanoparticles, wherein the solid nanoparticles comprise
i) an effective amount of a therapeutically active agent, wherein the therapeutically active agent is a substantially water insoluble prodrug; and
ii) a biocompatible polymer.

In another aspect, the invention provides a method of treating a disease or condition in a subject, comprising administering to the subject a pharmaceutical composition of the invention. In some embodiments, the disease or condition is cancer. In some embodiments, the cancer is selected from the group consisting of breast cancer, ovarian cancer, lung cancer, head and neck cancer, colon cancer, pancreatic cancer, melanoma, brain cancer, prostate cancer and renal cancer.

In another aspect, the invention provides a prodrug compound comprising everolimus conjugated to an omega-3 fatty acid. In some embodiments, the omega-3 fatty acid is selected from docosahexaenoic acid (DHA), eicosapentaenoic acid (EPA), and α-linolenic acid (LNA).

In another aspect, the invention provides a process for the preparation of a substantially stable dispersion of solid prodrug nanoparticles in an aqueous medium comprising:

combining (a) a first solution comprising a substantially water-insoluble prodrug, a water-immiscible organic solvent, and optionally a water-miscible organic solvent and with (b) an aqueous phase comprising water and an emulsifier, preferably a protein; forming an oil-in-water emulsion under high pressure homogenization and rapidly evaporating the water immiscible solvent under vacuum thereby producing solid prodrug nanoparticles stabilized by protein; wherein:

(i) the drugs are non-covalently encapsulated in the nanoparticles; wherein weak van der Waals' interactions exist between drug molecules;

(ii) wherein the nanoparticle formulation is capable of being sterile filtered and lyophilized;

(iii) wherein the lyophilized drug product is stable at refrigerated conditions or room temperature based on accelerated stability data.

In some embodiments, the process according to the present invention enables substantially stable dispersions of very small particles, especially nanoparticles, to be prepared in high concentration without particle growth.

The dispersion according to the present invention is substantially stable, by which is meant that the solid particles in the dispersion exhibit reduced or substantially no particle growth mediated by Ostwald ripening. By the term “reduced particle growth” is meant that the rate of particle growth mediated by Ostwald ripening is reduced compared to particles prepared without the use of an Ostwald ripening inhibitor. By the term “substantially no particle growth” is meant that the mean particle size of the particles in the aqueous medium does not increase by more than 20% (preferably by not more than 5% and more preferably <2%) over a period of 12-120 hours at 20° C. after the dispersion into the aqueous phase in the present process. By the term “substantially stable particle or nanoparticle” is meant that the mean particle size of the particles in the aqueous medium does not increase by more than 50% (more preferably by not more than 10%) over a period of 12-120 hours at 20° C. Preferably the particles exhibit substantially no particle growth over a period of 12-120 hours, more preferably over a period 24-120 hours and more preferably 48-120 hours.

It is to be understood that in those cases where the solid particles are prepared in an amorphous form the resulting particles will, generally, eventually revert to a thermodynamically more stable crystalline form upon storage as an aqueous dispersion. The time taken for such dispersions to re-crystallize is dependent upon the substance and may vary from a few hours to several days. Generally, such re-crystallization will result in particle growth and the formation of large crystalline particles which are prone to sedimentation from the dispersion. It is to be understood that the present invention does not prevent conversion of amorphous particles in the suspension into a crystalline state.

The solid particles in the dispersion preferably have a mean particle size of less than 10 μm, more preferably less than 5 μm, still more preferably less than 1 μm and especially less than 500 nm. It is especially preferred that the particles in the dispersion have a mean particle size of from 10 to 500 nm, more especially from 20 to 300 nm and still more especially from 20 to 200 nm. The mean size of the particles in the dispersion may be measured using conventional techniques, for example by dynamic light scattering to measure the intensity-averaged particle size. Generally, the solid particles in the dispersion prepared according to the present invention exhibit a narrow unimodal particle size distribution.

The solid particles may be crystalline, semi-crystalline or amorphous. In an embodiment, the solid particles comprise a pharmacologically active substance in a substantially amorphous form. This can be advantageous as many pharmacological compounds exhibit increased bioavailability in amorphous form compared to their crystalline or semi-crystalline forms. The precise form of the particles obtained will depend upon the conditions used during the evaporation step of the process. Generally, the present process results in rapid evaporation of the emulsion and the formation of substantially amorphous particles.

This invention provides a method for producing solid nanoparticles with mean diameter size of less than 220 nm, more preferably with a mean diameter size of about 20-200 nm and most preferably with a mean diameter size of about 20-180 nm. These solid nanoparticle suspensions can be sterile filtered through a 0.22 μm filter and lyophilized. The sterile suspensions can be lyophilized in the form of a cake in vials with or without cryoprotectants such as sucrose, mannitol, trehalose or the like. The lyophilized cake can be reconstituted to the original solid nanoparticle suspensions, without modifying the nanoparticle size, stability and the drug potency, and the cake is stable for more than 24 months.

In another embodiment, the sterile-filtered solid nanoparticles can be lyophilized in the form of a cake in vials using cryoprotectants such as sucrose, mannitol, trehalose or the like. The lyophilized cake can be reconstituted to the original particles, without modifying the particle size of solid nanoparticles. These nanoparticles are administered by a variety of routes, preferably by intravenous, parenteral, intratumoral and oral routes.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Chemical Structures of DHA-Cabazitaxel (Cabazitaxel Prodrug).

FIG. 2. Chemical Structures of DHA-Docetaxel (Docetaxel Prodrug).

FIG. 3. Chemical Structures of DHA-Everolimus (Everolimus Prodrug).

FIG. 4. The Particle Size Analysis of 4% Albumin after Homogenization with Chloroform and Ethanol.

FIG. 5. The Size Distribution of DHA-Cabazitaxel Nanoparticles Stabilized by Human Albumin (Lot PCD002).

FIG. 6. The Size Distribution of DHA-Everolimus Nanoparticles Stabilized by Human Albumin (Lot PED002).

FIG. 7. Stability of Reconstituted Nanoparticle Suspension of DHA-Cabazitaxel Stabilized by Human Albumin.

FIG. 8. Stability of Reconstituted Nanoparticle Suspension of DHA-Everolimus Stabilized by Human Albumin.

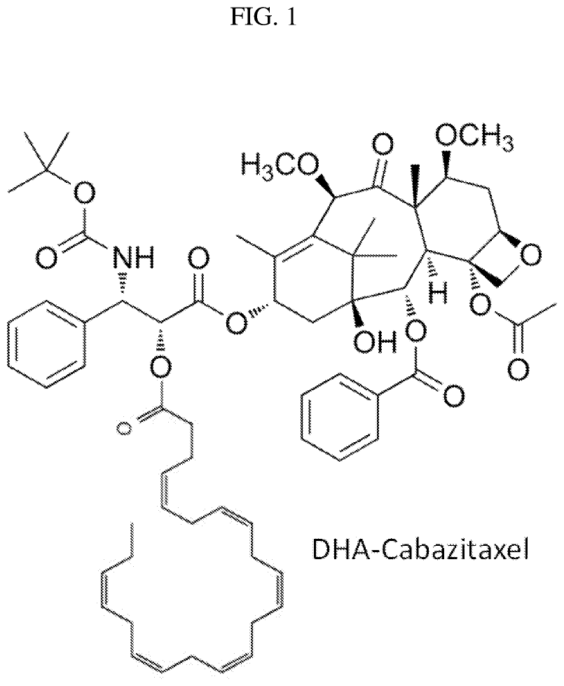

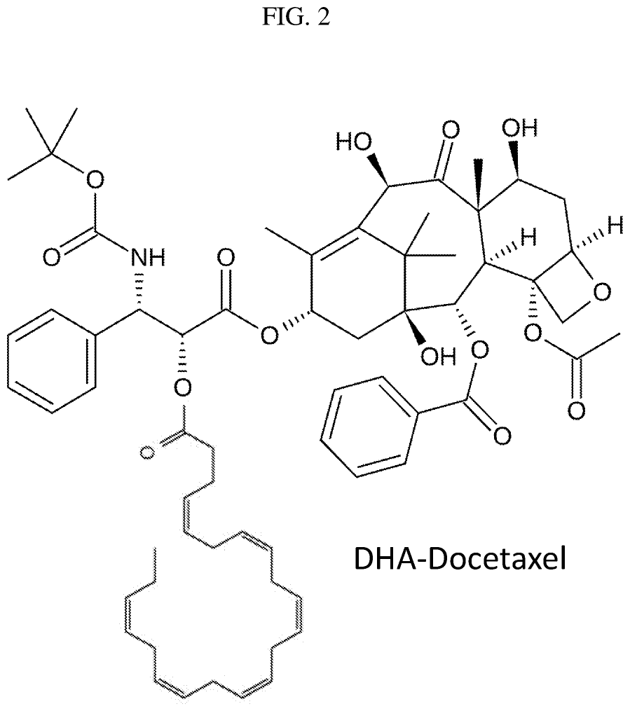

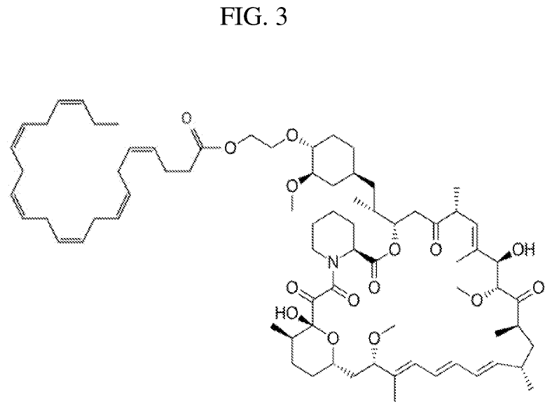

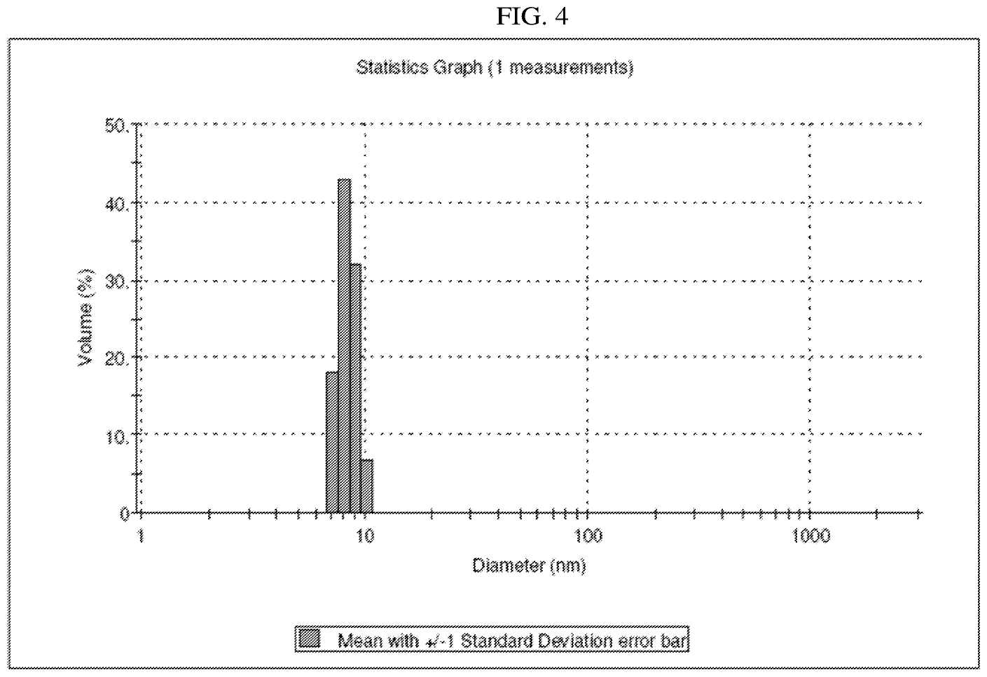

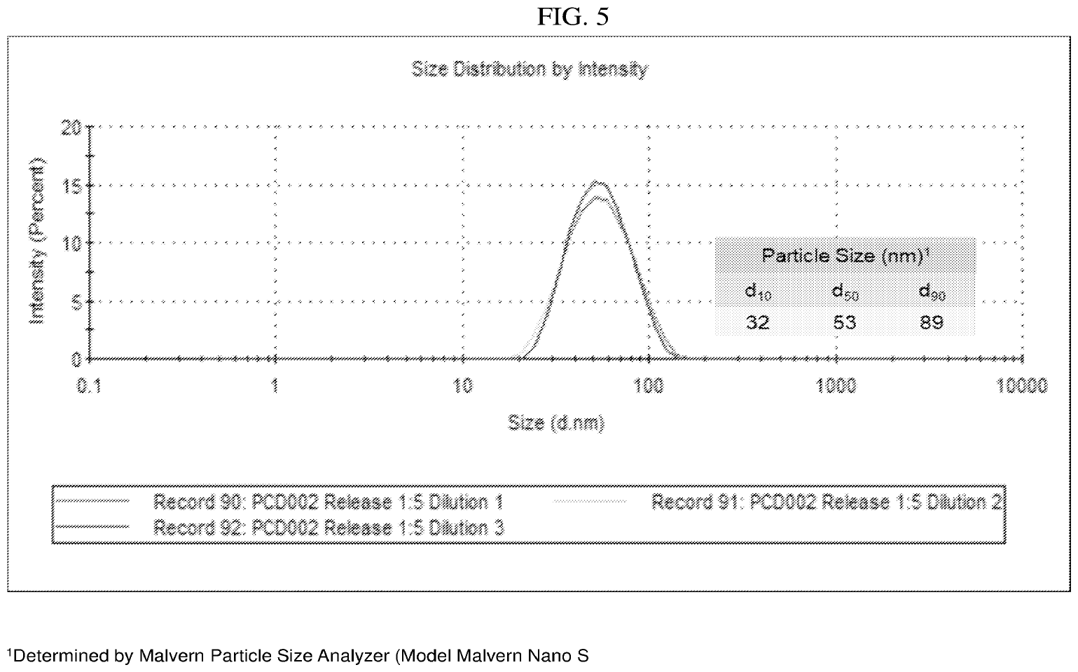

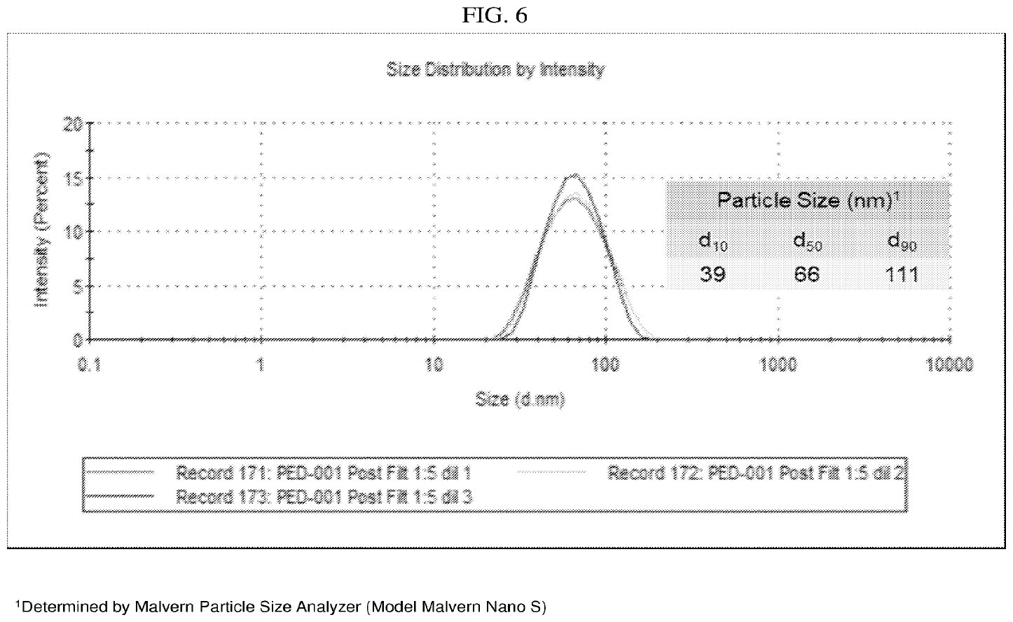

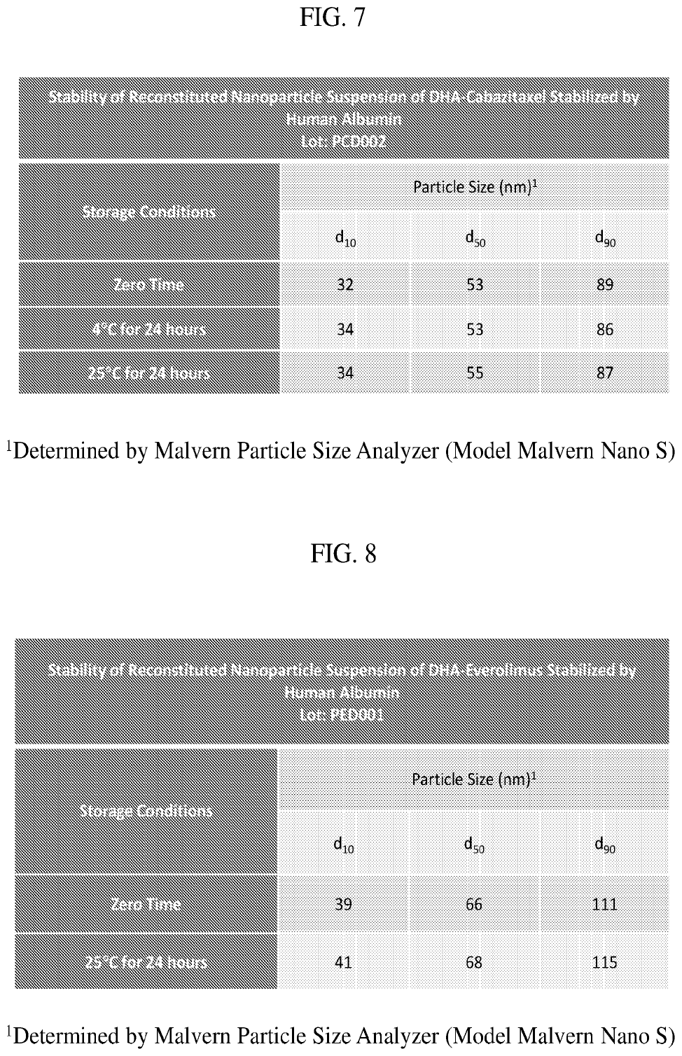

### DETAILED DESCRIPTION OF THE INVENTION

The compositions and methods of the present invention have distinct and surprising advantages over previously available compositions and methods. The prodrugs described herein are highly lipophilic and can be combined with human albumin by a suitable process, leading to the formation stable prodrug nanoparticles stabilized by human albumin.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

The term “Ostwald ripening” refers to coarsening of a precipitate or solid particle dispersed in a medium and is the final stage of phase separation in a solution, during which the larger particles of the precipitate or the solid particle grow at the expense of the smaller particles, which disappear. As recognized by Ostwald, the driving force for the process which now bears his name is the increased solubility of the smaller particles due to surface tension between the precipitate or the solid particle and the solute. If one assumes that the solute is in local equilibrium with the precipitate or the solid particle, then this solubility difference induces a solute concentration gradient and leads to a diffusive flux from the smaller to the larger particles. One speaks of diffusion-controlled growth (as opposed to growth controlled by slow deposition of solute atoms at the particle surfaces).

In some embodiments, the invention provides a composition comprising solid nanoparticles wherein the solid nanoparticles comprise

i) an effective amount of a therapeutically active agent, wherein the therapeutically active agent is a substantially water insoluble prodrug; and

ii) a biocompatible polymer.

As used herein, the terms “effective amount” or “therapeutically effective amount” are interchangeable and refer to an amount that results in an improvement or remediation of at least one symptom of the disease or condition. Those of skill in the art understand that the effective amount may improve the patient's or subject's condition, but may not be a complete cure of the disease and/or condition.

The term “preventing” as used herein refers to minimizing, reducing or suppressing the risk of developing a disease state or parameters relating to the disease state or progression or other abnormal or deleterious conditions.

The terms “treating” and “treatment” as used herein refer to administering to a subject a therapeutically effective amount of a composition so that the subject has an improvement in the disease or condition. The improvement is any observable or measurable improvement. Thus, one of skill in the art realizes that a treatment may improve the patient's condition, but may not be a complete cure of the disease. Treating may also comprise treating subjects at risk of developing a disease and/or condition.

In some embodiments, the compound(s) or composition(s) can be administered to the subject once, such as by a single injection or deposition at or near the site of interest. In some embodiments, the compound(s) or composition(s) can be administered to a subject over a period of days, weeks, months or even years. In some embodiments, the compound(s) or composition(s) is administered at least once a day to a subject. Where a dosage regimen comprises multiple administrations, it is understood that the effective amount of the compound(s) or composition(s) administered to the subject can comprise the total amount of the compound(s) or composition(s) administered over the entire dosage regimen.

In some embodiments, the prodrug of the invention comprises a drug (e.g., cabazitaxel, everolimus, docetaxel, and others) conjugated to an omega-3 fatty acid. Any omega-3 fatty acid can be used in accordance with the present invention. Examples of omega-3 fatty acids include docosahexaenoic acid (DHA), eicosapentaenoic acid (EPA), and α-linolenic acid (LNA). In some embodiments, the drug-conjugates (DHA-cabazitaxel, DHA-everolimus, DHA-docetaxel and others) of the present invention are useful for treating cancer in a human in need thereof. The cancer can be any type of cancer that is sensitive to docetaxel, cabazitaxel, everolimus, and others. Examples of cancers include breast, ovary, lung, head and neck, colon, pancreatic, melanoma, brain, prostate and renal cancer.

In some embodiments, the invention provides a prodrug compound comprising everolimus conjugated to an omega-3 fatty acid. In some embodiments, the omega-3 fatty acid is selected from docosahexaenoic acid (DHA), eicosapentaenoic acid (EPA), and α-linolenic acid (LNA).

In some embodiments, the invention provides a method comprises administering an effective amount of DHA-docetaxel or DHA-cabazitaxel or DHA-everolimus or others as nanoparticles stabilized by human albumin to a subject in need thereof. In some embodiments, an effective amount of DHA-docetaxel or DHA-cabazitaxel or DHA-everolimus or others is any amount effective in treating the cancer.

The advantages of these nanoparticle formulations are that substantially stable water insoluble prodrugs stabilized by human albumin are created with minimum or no Ostwald ripening. These compositions have been observed to provide a very low toxicity of the pharmacologically active agent that can be delivered in the form of nanoparticles or suspensions by slow infusions or by bolus injection or by other parenteral or oral delivery routes. In some embodiments, these nanoparticles have sizes below 400 nm, preferably below 200 nm, and more preferably below 140 nm having hydrophilic proteins adsorbed onto the surface of the nanoparticles. These nanoparticles can assume different morphologies; they can exist as amorphous particles or as crystalline particles.

By substantially insoluble is meant a substance that has a solubility in water at 25° C. of less than 0.5 mg/ml, preferably less than 0.1 mg/ml and especially less than 0.05 mg/ml.

The greatest effect on particle stability is observed when the substance has a solubility in water at 25° C. of less than 0.2 μg/ml. In a preferred embodiment the substance has a solubility in the range of from 0.001 μg/ml to 0.5 mg/ml.

In order to form the solid nanoparticles dispersed in an aqueous medium, in some embodiments, substantially water insoluble pharmaceutical prodrug substance and optionally an Ostwald ripening inhibitor(s) are dissolved in a suitable solvent (e.g., chloroform, methylene chloride, ethyl acetate, ethanol, tetrahydrofuran, dioxane, acetonitrile, acetone, dimethyl sulfoxide, dimethyl formamide, methyl pyrrolidinone, or the like, as well as mixtures of any two or more thereof).

In the next stage, in some embodiments, in order to make the solid nanoparticles, a protein (e.g., human serum albumin) is added (into the aqueous phase) to act as a stabilizing agent or an emulsifier for the formation of stable nanodroplets. Protein is added at a concentration in the range of about 0.05 to 25% (w/v), more preferably in the range of about 0.5%-10% (w/v).

In the next stage, in some embodiments, in order to make the solid nanoparticles, an emulsion is formed by homogenization under high pressure and high shear forces. Such homogenization is conveniently carried out in a high-pressure homogenizer, typically operated at pressures in the range of about 3,000 up to 30,000 psi. Preferably, such processes are carried out at pressures in the range of about 6,000 up to 25,000 psi. The resulting emulsion comprises very small nanodroplets of the nonaqueous solvent containing the substantially water insoluble pharmaceutical substance, optionally an Ostwald ripening inhibitor and other agents. Acceptable methods of homogenization include processes imparting high shear and cavitation such as high-pressure homogenization, high shear mixers, sonication, high shear impellers, and the like.

Finally, in some embodiments, in order to make the solid nanoparticles, the solvent is evaporated under reduced pressure to yield a colloidal system composed of solid nanoparticles of substantially water insoluble pharmaceutical prodrug substance and optionally an Ostwald ripening inhibitor(s) in solid form and protein. Acceptable methods of evaporation include the use of rotary evaporators, falling film evaporators, spray driers, freeze driers, and the like. Following evaporation of solvent, the liquid suspension may be dried to obtain a powder containing the pharmacologically active agent and protein. The resulting powder can be redispersed at any convenient time into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals. Methods contemplated for obtaining this powder include freeze-drying, spray drying, and the like.

In accordance with a specific embodiment of the present invention, there is provided a method for the formation of unusually small submicron solid particles containing substantially water insoluble pharmaceutical prodrug substance and optionally an Ostwald ripening inhibitor for Ostwald growth, i.e., particles which are less than 200 nanometers in diameter. Such particles are capable of being sterile-filtered before use in the form of a liquid suspension. The ability to sterile-filter the end product of the invention formulation process (i.e., the substantially water insoluble pharmaceutical substance particles) is of great importance since it is impossible to sterilize dispersions which contain high concentrations of protein (e.g., serum albumin) by conventional means such as autoclaving.

In some embodiments, in order to obtain sterile-filterable solid nanoparticles of substantially water insoluble pharmaceutical substances (i.e., particles <200 nm), the substantially water insoluble pharmaceutical prodrug substance and optionally an Ostwald ripening inhibitor(s) are initially dissolved in a substantially water immiscible organic solvent (e.g., a solvent having less than about 5% solubility in water, such as, for example, chloroform) at high concentration, thereby forming an oil phase containing the substantially water insoluble prodrug substance and optionally an Ostwald ripening inhibitor and other agents. Suitable solvents are set forth above. Next, a water miscible organic solvent (e.g., a solvent having greater than about 10% solubility in water, such as, for example, ethanol) is added to the oil phase at a final concentration in the range of about 1%-99% v/v, more preferably in the range of about 5%-25% v/v of the total organic phase. The water miscible organic solvent can be selected from such solvents as ethyl acetate, ethanol, tetrahydrofuran, dioxane, acetonitrile, acetone, dimethyl sulfoxide, dimethyl formamide, methyl pyrrolidinone, and the like. Alternatively, the mixture of water immiscible solvent with the water miscible solvent is prepared first, followed by dissolution of the substantially water insoluble pharmaceutical prodrug substance and optionally an Ostwald ripening inhibitor and other agents in the mixture. It is believed that the water miscible solvent in the organic phase act as a lubricant on the interface between the organic and aqueous phases resulting in the formation of fine oil in water emulsion during homogenization.

In the next stage, In some embodiments, for the formation of solid nanoparticles of substantially water insoluble pharmaceutical substances with reduced Ostwald growth, human serum albumin or any other suitable stabilizing agent as described above is dissolved in aqueous media. This component acts as an emulsifying agent for the formation of stable nanodroplets. Optionally, a sufficient amount of the first organic solvent (e.g.

chloroform) is dissolved in the aqueous phase to bring it close to the saturation concentration. A separate, measured amount of the organic phase (which now contains the substantially water insoluble pharmaceutical substances, the first organic solvent and the second organic solvent) is added to the saturated aqueous phase, so that the phase fraction of the organic phase is between about 0.5%-15% v/v, and more preferably between 1% and 8% v/v. Next, a mixture composed of micro and nanodroplets is formed by homogenization at low shear forces. This can be accomplished in a variety of ways, as can readily be identified by those of skill in the art, employing, for example, a conventional laboratory homogenizer operated in the range of about 2,000 up to about 15,000 rpm. This is followed by homogenization under high pressure (i.e., in the range of about 3,000 up to 30,000 psi). The resulting mixture comprises an aqueous protein solution (e.g., human serum albumin), the substantially water insoluble pharmaceutical prodrug substance and optionally an Ostwald ripening inhibitor(s), other agents, the first solvent and the second solvent. Finally, solvent is rapidly evaporated under vacuum to yield a colloidal dispersion system (solids of substantially water insoluble pharmaceutical prodrug substance and optionally an Ostwald ripening inhibitor and other agents and protein) in the form of extremely small nanoparticles (i.e., particles in the range of about 20 nm-200 nm diameter), and thus can be sterile-filtered. The preferred size range of the particles is between about 20 nm-170 nm, depending on the formulation and operational parameters.

In some embodiments, the solid nanoparticles prepared in accordance with the present invention may be further converted into powder form by removal of the water there from, e.g., by lyophilization at a suitable temperature-time profile. The protein (e.g., human serum albumin) itself acts as a cryoprotectant, and the powder is easily reconstituted by addition of water, saline or buffer, without the need to use such conventional cryoprotectants as mannitol, sucrose, trehalose, glycine, and the like. While not required, it is of course understood that conventional cryoprotectants may be added to invention formulations if so desired. The solid nanoparticles containing substantially water insoluble pharmaceutical substance allows for the delivery of high doses of the pharmacologically active agent in relatively small volumes.

According to this embodiment of the present invention, the solid nanoparticles containing substantially water insoluble pharmaceutical substance has a cross-sectional diameter of no greater than about 2 microns. A cross-sectional diameter of less than 1 microns is more preferred, while a cross-sectional diameter of less than 0.22 micron is presently the most preferred for the intravenous route of administration.

Proteins contemplated for use as stabilizing agents (biocompatible polymer) in accordance with the present invention include albumins (which contain 35 cysteine residues), immunoglobulins, caseins, insulins (which contain 6 cysteines), hemoglobins (which contain 6 cysteine residues per α2 β2 unit), lysozymes (which contain 8 cysteine residues), immunoglobulins, α-2-macroglobulin, fibronectins, vitronectins, fibrinogens, lipases, and the like. Proteins, peptides, enzymes, antibodies and combinations thereof, are general classes of stabilizers contemplated for use in the present invention.

A presently preferred protein for use is albumin. Human serum albumin (HSA) is the most abundant plasma protein (˜640 μM) and is non-immunogenic to humans. The protein is principally characterized by its remarkable ability to bind a broad range of hydrophobic small molecule ligands including fatty acids, bilirubin, thyroxine, bile acids and steroids; it serves as a solubilizer and transporter for these compounds and, in some cases, provides important buffering of the free concentration. HSA also binds a wide variety of drugs in two primary sites which overlap with the binding locations of endogenous ligands. The protein is a helical monomer of 66 kD containing three homologous domains (I-III) each of which is composed of A and B subdomains. The measurements on erythrosin-bovine serum albumin complex in neutral solution, using the phosphorescence depolarization techniques, are consistent with the absence of independent motions of large protein segments in solution of BSA, in the time range from nanoseconds to fractions of milliseconds. These measurements support a heart shaped structure (8 nm×8 nm×8 nm×3.2 nm) of albumin in neutral solution of BSA as in the crystal structure of human serum albumin. Another advantage of albumin is its ability to transport drugs into tumor sites. Specific antibodies may also be utilized to target the nanoparticles to specific locations. HSA contains only one free sulfhydryl group as the residue Cys34 and all other Cys residues are bridged with disulfide bonds (Sugio S, et al., Crystal structure of human serum albumin at 2.5 A resolution. Protein Eng 1999; 12: 439-446).

In the preparation of the inventive compositions, a wide variety of organic media can be employed to dissolve the substantially water insoluble pharmaceutical substances.

Especially preferred combinations of organic media contemplated for use in the practice of the present invention typically have a boiling point of no greater than about 200° C., and include volatile liquids such as dichloromethane, chloroform, ethyl acetate, benzene, and the like (i.e., solvents that have a high degree of solubility for the pharmacologically active agent, and are soluble in the other organic medium employed), along with a higher molecular weight (less volatile) organic medium. When added to the other organic medium, these volatile additives help to drive the solubility of the pharmacologically active agent into the organic medium. This is desirable since this step is usually time consuming. Following dissolution, the volatile component may be removed by evaporation (optionally under vacuum).

The solid nanoparticle formulations prepared in accordance with the present invention may further contain certain chelating agents. The biocompatible chelating agent to be added to the formulation can be selected from ethylenediaminetetraacetic acid (EDTA), diethylenetriaminepentaacetic acid (DTPA), ethylene glycol-bis(β-aminoethyl ether)-tetraacetic acid (EGTA), N-(hydroxyethyl)-ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

The nanoparticle formulations prepared in accordance with the present invention may further contain certain antioxidants which can be selected from ascorbic acid derivatives such as ascorbic acid, erythorbic acid, sodium ascorbate, ascorbyl palmitate, retinyl palmitate; thiol derivatives such as thioglycerol, cysteine, acetylcysteine, cystine, dithioerythreitol, dithiothreitol, gluthathione; tocopherols; propyl gallate, butylated hydroxyanisole; butylated hydroxytoluene; sulfurous acid salts such as sodium sulfate, sodium bisulfite, acetone sodium bisulfite, sodium metabisulfite, sodium sulfite.

The nanoparticle formulations prepared in accordance with the present invention may further contain certain preservatives if desired. The preservative for adding into the present inventive formulation can be selected from phenol, chlorobutanol, benzoic acid, sodium benzoate, benzyl alcohol, methylparaben, propylparaben, benzalkonium chloride and cetylpyridinium chloride.

The solid nanoparticles containing substantially water insoluble pharmaceutical prodrug substance and optionally an Ostwald ripening inhibitor with protein, prepared as described above, can be delivered as a suspension in a biocompatible aqueous liquid. This liquid may be selected from water, saline, a solution containing appropriate buffers, a solution containing nutritional agents such as amino acids, sugars, proteins, carbohydrates, vitamins or fat, and the like.

For increasing the long-term storage stability, the solid nanoparticle formulations may be frozen and lyophilized in the presence of one or more protective agents such as sucrose, mannitol, trehalose or the like. Upon rehydration of the lyophilized solid nanoparticle formulations, the suspension retains essentially all the substantially water insoluble pharmaceutical substance previously loaded and the particle size. The rehydration is accomplished by simply adding purified or sterile water or 0.9% sodium chloride injection or 5% dextrose solution followed by gentle swirling of the suspension. The potency of the substantially water insoluble pharmaceutical substance in a solid nanoparticle formulation is not lost after lyophilization and reconstitution.

In some embodiments, the solid nanoparticle formulations of the present invention are shown to be less prone to Ostwald ripening due to the modification of the parent drug molecule to make the prodrug, and optionally, addition of one or more Ostwald ripening inhibitors and are more stable in solution than the formulations disclosed in the prior art. In the present invention, efficacy of solid nanoparticle formulations of the present invention with varying Ostwald ripening inhibitor compositions, particle size, and substantially water insoluble pharmaceutical substance to protein ratio have been investigated on various systems such as human cell lines and animal models for cell proliferative activities.

In some embodiments, the solid nanoparticle formulation of the present invention is shown to be less toxic than the substantially water insoluble pharmaceutical substance administered in its free form. Furthermore, effects of the solid nanoparticle formulations and various substantially water insoluble pharmaceutical substances in their free form on the body weight of mice with different sarcomas and healthy mice without tumor have been investigated.

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier. In some embodiments, the disease or condition to be treated is cancer.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate;

powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

In some embodiments, the total daily dose of the active compounds of the present invention administered to a subject in single or in divided doses can be in amounts, for example, from 0.01 to 25 mg/kg body weight or more usually from 0.1 to 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

The active agents of the present invention can be administered alone or in combination with one or more active pharmaceutical agents or treatments. In some embodiments, the one or more active pharmaceutical agents are useful to treat cancer in the subject. Additional treatments can include typical treatments for cancer, such as surgery, radiation, and the like.

Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulation can be sterilized, for example, by filtration through a bacteria-retaining filter, or by incorporating sterilizing agents in the form of sterile solid compositions, which can be dissolved or dispersed in sterile water or other sterile injectable medium just prior to use.

Suppositories for rectal administration of the drug can be prepared by mixing the drug with a suitable non-irritating excipient, such as cocoa butter and polyethylene glycol which are solid at ordinary temperature but liquid at the rectal temperature and will, therefore, melt in the rectum and release the drug.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In one embodiment, the therapeutic compound is delivered transdermally. The term “transdermal delivery” as used herein means administration of the pharmaceutical composition topically to the skin wherein the active ingredient or its pharmaceutically acceptable salts, will be percutaneously delivered in a therapeutically effective amount.

In some embodiments, the composition to be applied transdermally further comprises an absorption enhancer. The term “absorption enhancer” as used herein means a compound which enhance the percutaneous absorption of drugs. These substances are sometimes also referred to as skin-penetration enhancers, accelerants, adjuvants and sorption promoters. Various absorption enhancers are known to be useful in transdermal drug delivery. U.S. Pat. Nos. 5,230,897, 4,863,970, 4,722,941, and 4,931,283 disclose some representative absorption enhancers used in transdermal compositions and for topical administration. In some embodiments, the absorption enhancer is N-lauroyl sarcosine, sodium octyl sulfate, methyl laurate, isopropyl myristate, oleic acid, glyceryl oleate or sodium lauryl sulfoacetate, or a combination thereof. In some embodiments, the composition contains on a weight/volume (w/v) basis the absorption enhancer in an amount of about 1-20%, 1-15%, 1-10% or 1-5%. In some embodiments, to enhance further the ability of the therapeutic agent(s) to penetrate the skin or mucosa, the composition can also contain a surfactant, an azone-like compound, an alcohol, a fatty acid or ester, or an aliphatic thiol.

In some embodiments, the transdermal composition can further comprise one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

The method of the present invention employs the compounds identified herein for both in vitro and in vivo applications. For in vivo applications, the invention compounds can be incorporated into a pharmaceutically acceptable formulation for administration. Those of skill in the art can readily determine suitable dosage levels when the invention compounds are so used.

Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition calculated to produce the desired responses in association with its administration, e.g., the appropriate route and treatment regimen. The quantity to be administered, and the particular route and formulation, are within the skill of those in the clinical arts. Also of importance is the subject to be treated, in particular, the state of the subject and the protection desired. A unit dose need not be administered as a single injection but may comprise continuous infusion over a set period of time.

The examples provided here are not intended, however, to limit or restrict the scope of the present invention in any way and should not be construed as providing conditions, parameters, reagents, or starting materials which must be utilized exclusively in order to practice the art of the present invention.

### EXAMPLES

### Example 1. Effect of Emulsification on Human Serum Albumin

An organic phase was prepared by mixing 3.5 mL of chloroform and 0.6 mL of dehydrated ethanol. A 4% human albumin solution was prepared by dissolving 2 gm of human albumin (Sigma-Aldrich Co, USA) in 50 mL of sterile Type I water. The pH of the human albumin solution was adjusted to 6.0-6.7 by adding either 1N hydrochloric acid or 1N sodium hydroxide solution in sterile water. The above organic solution was added to the albumin phase and the mixture was pre-homogenized with an IKA homogenizer at 6000-10000 RPM (IKA Works, Germany). The resulting emulsion was subjected to high-pressure homogenization (Avestin Inc, USA). The pressure was varied between 20,000 and 30,000 psi and the emulsification process was continued for 5-8 passes. During homogenization the emulsion was cooled between 5° C. and 10° C. by circulating the coolant through the homogenizer from a temperature-controlled heat exchanger (Julabo, USA). This resulted in a homogeneous and extremely fine oil-in-water emulsion. The emulsion was then transferred to a rotary evaporator (Buchi, Switzerland) and rapidly evaporated to obtain an albumin solution subjected to high pressure homogenization. The evaporator pressure was set during the evaporation by a vacuum pump (Welch) at 1-5 mm Hg and the bath temperature during evaporation was set at 35° C.

The particle size of the albumin solution was determined by photon correlation spectroscopy with a Malvern Zetasizer. It was observed that there were two peaks, one around 5-8 nm and other around 120-140 nm. The peak around 5-8 nm contained nearly 99% by volume and the peak around 120-140 nm had less than 1% by volume (FIG. 9). As a control, the particle size distribution in 4% human serum solution was measured. It had only one peak around 5-8 nm (FIG. 10). These studies show that the homogenization of an albumin solution in an oil-in-water emulsion renders less than 2-3 percent of the albumin molecules to be aggregated by denaturation.

### Example 2. Preparation of Unstable Solid Cabazitaxel Nanoparticles

An organic solution was prepared by dissolving 600 mg of Cabazitaxel (Polymed Therapeutics, TX, USA) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution was approximately 7.0 and was used without further pH adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 24 mm Hg, set by a vacuum pump (Leybold USA, Inc., PA, USA), and the bath temperature maintained at 35° C.

An off-white slightly translucent suspension with a small amount of visible solid particulate was obtained. The particle size of the suspension was determined by laser diffraction with a Particle Size Analyzer (Beckman Coulter Life Sciences, IN, USA) and found to have formed nanoparticles with a size distribution between 59 and 114 nm (dio and d₉₀, respectively) with a d₅₀ size of 83 nm. The suspension was divided into aliquots and stored at refrigerated and room temperatures; after 24 hours both samples showed a small amount of fine precipitate had sedimented on bottom of the containers. Particle size analysis of both samples showed similar distributions between 61 and 129 nm (d₁₀ and d₉₀, respectively) with a d₅₀ size of 88 nm. The d₉₉ particle size after 24 hours had changed from 142 nm to 164 nm. The formulation containing the above composition was designated as unstable due to Ostwald ripening and therefore not suitable for sterile filtration and further development.

### Example 3. Preparation of Stable Solid Nanoparticles of DHA-Cabazitaxel

An organic phase was prepared by dissolving 796 mg of DHA-Cabazitaxel (Rational Labs Pvt. Ltd., Hyderabad, Telgana, India) in an inert Nitrogen atmosphere (Matheson Tri-Gas, TX, USA) in a mixture of 3.15 mL of Chloroform (Spectrum Chemical, NJ, USA) and 0.35 mL of anhydrous Ethanol (Spectrum Chemical, NJ, USA), in which the solvents were previously sparged with Nitrogen gas. A 5% human albumin solution was prepared by diluting 9.3 mL of 25% human albumin (Grifols Biologicals, Inc., CA, USA) in 37.2 mL of Water for Injection (Rocky Mountain Biologicals, UT, USA), in which these materials were vacuum degassed and sparged with Nitrogen gas, respectively.

The above organic solution was added to the albumin phase and the mixture was pre-homogenized with a high shear homogenizer at 10,000 RPM (IKA Works, Inc., NC, USA) under a Nitrogen bed. The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water, and in which the process stream was kept under a positive pressure Nitrogen bed. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A light yellow very translucent suspension was obtained and determined by HPLC assay (Waters Corp., MA, USA) to be 14.5 mg/mL which was then diluted to 7.0 mg/mL with 25% human albumin and water for injection to make 5% human albumin in the final product. The diluted suspension was serially sterile-filtered through 0.45 μm and then 0.22 μm filter units (Celltreat Scientific Products, MA, USA). A light yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 48 nm with a polydispersity index of 0.164. Vials were filled with a volume equivalent to 10 mg Docosahexenoate Cabazi and lyophilized. A vial was reconstituted with water to 5 mg/mL and the particle size was found to have a Z-average of 48 nm with a polydispersity index of 0.167. Aliquots of the suspension were held at 4° C. and 25° C. for 24 hours with Z-average sizes and polydispersities of 48 nm (0.161) and 50 nm (0.144), respectively.

### Example 4. Preparation of Unstable Solid Everolimus Nanoparticles

An organic solution was prepared by dissolving 601 mg of Everolimus (Bright Gene Biomedical Tech Co. Ltd., Suzhou, China) in a mixture of 2.7 mL of Chloroform (Spectrum Chemical, NJ, USA) and 0.3 mL of anhydrous Ethanol (Spectrum Chemical, NJ, USA). A 5% human albumin solution was prepared by diluting 9.4 mL of 25% human albumin (Grifols Biologicals, Inc., CA, USA) in 37.6 mL of Water for Injection (Rocky Mountain Biologicals, UT, USA). The pH of the albumin solution is approximately 7.3 and is used without adjustment.

The above organic solution was added to the albumin phase and the mixture was pre-homogenized with a high shear homogenizer at 10,000 RPM (IKA Works, Inc., NC, USA). The crude emulsion was then subjected to high-pressure homogenization (Microfluidics Corp., MA, USA) at 20,000 psi for 4 passes, recycling the emulsion into the process stream after cooling to about 2-4° C. by passing the fluidic path tubing through an ice bath. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 22 mm Hg, set by a vacuum pump (Leybold USA, Inc., PA, USA), and the bath temperature maintained at 35° C.

An off-white slightly translucent suspension containing large amounts of visible particulate solids was obtained. The particle size of the suspension was determined by laser diffraction with a Particle Size Analyzer (Beckman Coulter Life Sciences, IN, USA) and found to have formed nanoparticles with a size distribution between 96 and 157 nm (d₁₀ and d₉₀, respectively) with a d₅₀ size of 123 nm. The suspension was divided into aliquots and stored at refrigerated conditions and room temperature; after 24 hours both samples showed visible precipitate had sedimented on bottom of the containers. Particle size analysis of both samples showed similar distributions between 77 and 264 nm (d₁₀ and d₉₀) with a d₅₀ size of 138 nm. The d₉₉ particle size after 24 hours had changed from 188 nm to 427 nm. The formulation containing the above composition was designated as unstable due to Ostwald ripening and therefore not suitable for sterile filtration and further development.

### Example 5. Preparation of Stable Solid Nanoparticles of DHA-Everolimus

An organic phase was prepared by dissolving f 407 mg of DHA-Everolimus (Rational Labs Pvt. Ltd., Hyderabad, Telgana, India) in an inert Nitrogen atmosphere (Matheson Tri-Gas, TX, USA) in mixture of 1.8 mL of Chloroform (Spectrum Chemical, NJ, USA) and 0.2 mL of anhydrous Ethanol (Spectrum Chemical, NJ, USA), in which the solvents were previously sparged with Nitrogen gas. A 5% human albumin solution was prepared by diluting 9.6 mL of 25% human albumin (Grifols Biologicals, Inc., CA, USA) in 38.4 mL of Water for Injection (Rocky Mountain Biologicals, UT, USA), in which these materials were vacuum degassed and sparged with Nitrogen gas, respectively.

The above organic solution was added to the albumin phase and the mixture was pre-homogenized with a high shear homogenizer at 10,000 RPM (IKA Works, Inc., NC, USA) under a Nitrogen bed. The crude emulsion was then subjected to high-pressure homogenization (Microfluidics Corp., MA, USA) at 20,000 psi for 4 passes, recycling the emulsion into the process stream after cooling to about 4° C. by passing through a heat exchange coil submerged in ice water, and in which the process stream was kept under a positive pressure Nitrogen bed. This resulted in a homogeneous and extremely fine oil-in-water emulsion that was collected and transferred at once to a rotary evaporator (Yamato Scientific America, Inc., CA, USA) and rapidly evaporated to a nanoparticle suspension at an initial pressure of 27 mm Hg, set by a vacuum pump (Leybold USA, Inc., PA, USA), and the bath temperature maintained at 40° C.

A yellow very translucent suspension was obtained and determined by HPLC assay (Waters Corp., MA, USA) to be 5.1 mg/mL which was then sterile-filtered without dilution through a 1.0 μm prefilter and 0.22 μm filter unit (Celltreat Scientific Products, MA, USA). A yellow, very translucent, particulate-free suspension was obtained. The particle size of the suspension was determined by photo correlation spectroscopy with a Zetasizer Nano (Malvern Panalytical, MA, USA) and found to have formed nanoparticles with a Z-average size of 58 nm with a polydispersity index of 0.178. A sample kept at room temperature (20-25° C.) for 24 hours was found to have a Z-average size of 62 nm and polydispersity index of 0.165.

1. A pharmaceutical composition comprising solid nanoparticles, wherein the solid nanoparticles comprise
i) an effective amount of a therapeutically active agent, wherein the therapeutically active agent is a substantially water insoluble prodrug; and
ii) a biocompatible polymer.

2. The pharmaceutical composition of claim 1, wherein the composition comprises a substantially stable and sterile filterable dispersion of solid nanoparticles in an aqueous medium, wherein the solid nanoparticles comprise the substantially water insoluble prodrug or a mixture thereof and have a mean particle size of less than 220 nm as measured by particle size analyzer, wherein the composition is prepared by a process comprising:
(a) combining an aqueous phase comprising water and a biocompatible polymer as emulsifier and an organic phase comprising the water insoluble prodrug undergoing little or no Ostwald ripening, a water-immiscible organic solvent, optionally a water-miscible organic solvent as an interfacial lubricant;
(b) forming an oil-in-water emulsion using a high-pressure homogenizer;
(c) removing the water-immiscible organic solvent and the water-miscible organic solvent from the oil-in water emulsion under vacuum, thereby forming a substantially stable dispersion of solid nanoparticles comprising the biocompatible polymeric emulsifier and the water insoluble prodrug drug undergoing little or no Ostwald ripening in the aqueous medium.

3. The pharmaceutical composition according to claim 2, wherein the substantially water insoluble prodrug is selected from the parent molecules including cabazitaxel, everolimus, docetaxel, and similar taxanes.

4. The pharmaceutical composition according to claim 2, wherein the substantially water insoluble prodrug is selected from the parent molecules including camptothecins (topotecan, irinotecan, SN-38, S39625, and S38809), doxorubicin, eribulin, rapamycin, cytarabine, etoposide, podophyllotoxin, temozolomide, methotrexate, floxuridine, gemcitabine, mitomycin, riluzole, cladribine, melphalan, cidofovir, fulvestrant, melphalan, cannabinoids (cannabidiol, tetrahydrocannabinol, cannabinol, cannabigerol, tetrahydrocannabinolic acid, cannabidiolic acid, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran), aprepitant, morphine, and hydrocodone.

5. The pharmaceutical composition according to claim 4, wherein said biocompatible polymer is human albumin or recombinant human albumin or PEG-human albumin

6. The pharmaceutical composition according to claim 5, further comprising a pharmaceutically acceptable preservative or mixture thereof, wherein said preservative is selected from the group consisting of phenol, chlorobutanol, benzylalcohol, methylparaben, propylparaben, benzalkonium chloride and cetylpyridinium chloride.

7. The pharmaceutical composition according to claim 6, further comprising a biocompatible chelating agent wherein said biocompatible chelating agent is selected from the group consisting of ethylenediaminetetraacetic acid (EDTA), diethylenetriaminepentaacetic acid (DTPA), ethylene glycol-bis(β-aminoethyl ether)-tetraacetic acid (EGTA), N(hydroxyethyl) ethylenediaminetriacetic acid (HEDTA), nitrilotriacetic acid (NTA), triethanolamine, 8-hydroxyquinoline, citric acid, tartaric acid, phosphoric acid, gluconic acid, saccharic acid, thiodipropionic acid, acetonic dicarboxylic acid, di(hydroxyethyl)glycine, phenylalanine, tryptophan, glycerin, sorbitol, diglyme and pharmaceutically acceptable salts thereof.

8. The pharmaceutical composition according to claim 7, further comprising an antioxidant, wherein said antioxidant is selected from the group consisting of ascorbic acid, erythorbic acid, sodium ascorbate, thioglycerol, cysteine, acetylcysteine, cystine, dithioerythreitol, dithiothreitol, gluthathione, tocopherols, butylated hydroxyanisole, butylated hydroxytoluene, sodium sulfate, sodium bisulfite, acetone sodium bisulfite, sodium metabisulfite, sodium sulfite, sodium formaldehyde sulfoxylate, sodium thiosulfate, and nordihydroguaiaretic acid.

9. The pharmaceutical composition according to claim 8, further comprising a buffer.

10. The pharmaceutical composition according to claim 9, further comprising a cryoprotectant selected from the group consisting of mannitol, sucrose and trehalose.

11. The pharmaceutical composition according to claim 10, wherein the aqueous medium containing the solid nanoparticle is sterilized by filtering through a 0.22-micron filter.

12. The pharmaceutical composition of claim 11, wherein the pharmaceutical composition is freeze-dried or lyophilized.

13. The pharmaceutical composition of claim 12, wherein the prodrug is conjugated to an omega-3 fatty acid.

14. The pharmaceutical composition of claim 13, wherein the prodrug is conjugated to an omega-3 fatty acid selected from the group consisting of docosahexaenoic acid (DHA), eicosapentaenoic acid (EPA), and α-linolenic acid (LNA).

15. The pharmaceutical composition of claim 14, wherein the prodrug is selected from the group consisting of DHA-cabazitaxel, DHA-everolimus, DHA-docetaxel and a combination thereof.

16. A method of treating a disease or condition in a subject, comprising administering to the subject the pharmaceutical composition of claim 15.

17. The method of claim 16, wherein the disease or condition is cancer.

18. The method of claim 17, wherein the cancer is selected from the group consisting of breast cancer, ovarian cancer, lung cancer, head and neck cancer, colon cancer, pancreatic cancer, melanoma, brain cancer, prostate cancer and renal cancer.

19. A prodrug compound comprising everolimus conjugated to an omega-3 fatty acid.

20. The prodrug of claim 19, wherein the omega-3 fatty acid is selected from docosahexaenoic acid (DHA), eicosapentaenoic acid (EPA), and α-linolenic acid (LNA).

21. (canceled)
